(19) United States
(12) Patent Application Publication
(10) Pub. No.: US 2026/0004910 A1
(43) Pub. Date: Jan. 1, 2026

(54) MEDICAMENT DELIVERY DEVICE WITH AN ADJUSTABLE AND PIECEWISE ANALYTE LEVEL COST COMPONENT TO ADDRESS PERSISTENT POSITIVE ANALYTE LEVEL EXCURSIONS

(71) Applicant: INSULET CORPORATION, Acton, MA (US)

(72) Inventors: Joon Bok LEE, Acton, MA (US); Eric BENJAMIN, Cambridge, MA (US); Jason O'CONNOR, Acton, MA (US); Yibin ZHENG, Hartland, WI (US)

(21) Appl. No.: 19/317,933

(22) Filed: Sep. 3, 2025

Related U.S. Application Data
(63) Continuation of application No. 17/691,829, filed on Mar. 10, 2022, now Pat. No. 12,431,229.
(60) Provisional application No. 63/158,918, filed on Mar. 10, 2021.

(51) Int. Cl. G16H 20/13; A61M 5/172

(52) U.S. Cl. CPC G16H 20/13; A61M 5/1723; A61M 2205/52; A61M 2230/201

(57) ABSTRACT

The exemplary embodiments may modify a glucose cost component of the cost function of the control loop of an insulin delivery device to compensate for persistent positive low level glucose excursions relative to a target glucose level. The exemplary embodiments may enable use of different glucose cost component functions for different glucose levels of the user. These glucose cost component functions may be employed in piecewise fashion with a different piece being applied for each respective range of glucose level values for the user. The final glucose cost function for calculating the glucose cost component may be a weighted combination of a piecewise glucose cost function and a weighted standard cost function (such as a quadratic function). The weights may reflect the magnitude and/or persistence of glucose excursions relative to a target glucose level.

### RELATED APPLICATIONS

This application is a continuation of U.S. patent application Ser. No. 17/691,829, filed Mar. 10, 2022, which claims the benefit of U.S. Provisional Patent Application No. 63/158,918, filed Mar. 10, 2021, the contents of which are incorporated herein by reference in their entirety.

### BACKGROUND

Some insulin pump systems may use a closed loop control system for regulating the amount of insulin delivered at fixed intervals, such as every 5 minutes. The closed loop algorithms used by the control system may employ a penalty for large insulin deliveries that is balanced in a cost function with a penalty for glucose level excursions. The use of the cost function typically results in smaller automated insulin deliveries that are delivered more frequently than with manually delivered boluses. The closed loop system may reassess a patient's need more often than a manual approach.

### SUMMARY

In accordance with an inventive facet disclosed herein, a diabetic treatment medicament delivery device includes a non-transitory storage medium for storing computer programming instructions for controlling operation of the diabetic treatment medicament delivery device. The diabetic treatment medicament delivery device also includes a processor for executing the computer programming instructions to cause the processor to determine a selected basal diabetic treatment medicament dosage to be delivered by the diabetic treatment medicament delivery device to a user from among candidate basal diabetic treatment medicament dosages. The determining includes determining the selected basal diabetic treatment medicament dosage based upon costs of the candidate basal diabetic treatment medicament dosages. The cost is determined by a cost function for determining a cost for each of the candidate basal diabetic treatment medicament dosages, where, for each of the candidate basal diabetic treatment medicament dosages, the cost function includes a glucose cost component that punishes glucose excursions relative to a target glucose level that are anticipated to be experienced by the user if the candidate basal diabetic treatment medicament dosage is delivered to the user by the diabetic treatment medicament delivery device. The glucose cost component varies in accordance with a first distribution function when the anticipated glucose excursions to be experienced by the user are in a first range and varies in accordance with a second distribution function when the anticipated glucose excursions to be experienced by the user are in a second range that differs from the first range. The computer programming instructions also cause the processor to cause the determined basal diabetic treatment medicament dosage to be delivered from the diabetic treatment medicament delivery device to the user.

The glucose cost component may be a combination of a piecewise cost function and an additional cost function. The weights assigned to the piecewise cost function and the additional cost function in the glucose cost component may depend on the persistence and the magnitude of glucose excursions experienced by the user. The first distribution function may be a quadratic function. The second distribution function may be a linear function. The first distribution function may be an exponential function or a logarithmic function. The diabetic treatment medicament may include at least one of insulin, a glucagon-like peptide-1 (GLP-1) agonist, pramlintide, or a co-formulation of two of the foregoing. The cost function also may include a diabetic treatment medicament cost component that represents a penalty based on an amount of diabetic treatment medicament in each of the candidate basal diabetic treatment medicament dosages.

In accordance with another inventive facet disclosed herein, a medicament delivery device includes a non-transitory storage medium for storing computer programming instructions for controlling operation of the medicament delivery device. The device also includes a processor for executing the computer programming instructions as to cause the processor to determine a selected basal medicament dosage to be delivered by the medicament delivery device to a user from among candidate basal medicament dosages such that the determining the selected basal medicament dosage is based upon costs of the candidate basal medicament dosages. The cost is determined by a cost function for determining a cost for each of the candidate basal medicament dosages, where, for each of the candidate basal medicament dosages, the cost function includes an analyte level component that punishes analyte level excursions relative to a target analyte level that are anticipated to be experienced by the user if the candidate basal medicament dosage is delivered to the user by the medicament delivery device. The analyte cost component varies in accordance with a first distribution function when the anticipated analyte level excursions to be experienced by the user are in a first range and varies in accordance with a second distribution function when the anticipated analyte level excursions to be experienced by the user are in a second range that differs from the first range. The computer programming instructions also cause the processor to cause the determined basal medicament dosage to be delivered from the medicament delivery device to the user.

The analyte cost component may be a combination of a piecewise cost function and an additional cost function. The weights assigned to the piecewise cost function and the additional cost function in the analyte level cost component may depend on the persistence and the magnitude of analyte excursions experienced by the user. The first distribution function may be one of a quadratic function, an exponential function, a linear function, or a logarithmic function. The cost function also may include a medicament cost component that represents a penalty based on an amount of medicament in each of the candidate basal diabetic treatment medicament dosages.

In accordance with an additional inventive facet disclosed herein, a method is performed by a processor for controlling basal medicament deliveries by a medicament delivery device. The method includes, with the processor, determining a medicament cost for a candidate basal medicament dosage for delivery to a user by the medicament delivery device and determining an analyte level cost for the candidate basal dosage. The analyte level cost includes a piecewise cost function that uses a first function to calculate analyte level cost for a first range of analyte level values, a second function to calculate analyte level cost for a second range of analyte level values, an additional cost function, a weight applied to the piecewise cost function, and a weight applied to the additional cost function. Per the method, a cost for the candidate basal medicament dosage is determined with the processor using the medicament cost and the analyte level cost, and based on the cost, a decision whether to deliver the candidate basal medicament dosage is made.

The medicament may be insulin. The method may include calculating the weight applied to the piecewise function from historical glucose levels. Calculating the weight applied to the piecewise function may entail calculating an offset value that captures how often there are positive glucose excursions and how often the positive glucose excursions occur in the historical glucose levels. The weight applied to the additional function may be (1−the offset value). The piecewise function may be a combination of a quadratic function and a linear function.

### BRIEF DESCRIPTION OF THE DRAWINGS

FIG. 1 an illustrative medicament delivery system suitable for the exemplary embodiments.

FIG. 2 depicts an illustrative control loop of exemplary embodiments for adjusting a medicament delivery dosage.

FIG. 3 depicts a flowchart of illustrative steps that may be performed in a control loop of exemplary embodiments.

FIG. 4 depicts an illustrative quadratic glucose cost function.

FIG. 5 depicts an illustrative linear glucose cost function.

FIG. 6 depicts an illustrative piecewise glucose cost function that may be used in exemplary embodiments.

FIG. 7 depicts a flowchart of illustrative steps that may be performed in exemplary embodiments to use different functions in a piecewise cost function.

FIG. 8 depicts a flowchart of illustrative steps that may be performed in exemplary embodiments to combine a quadratic function and a linear function in a piecewise glucose cost function.

FIG. 9 depicts a flowchart of illustrative steps that may be performed in exemplary embodiments to calculate a quadratic glucose cost function value.

FIG. 10 depicts a flowchart of illustrative steps that may be performed in exemplary embodiments to calculate a linear glucose cost function value.

FIG. 11 depicts a flowchart of illustrative steps that may be performed in exemplary embodiments to calculate a final glucose cost function value.

FIG. 12 depicts a flowchart of illustrative steps that may be performed in exemplary embodiments to calculate an offset value from historical glucose level data.

FIG. 13 depicts a flowchart of illustrative steps that may be performed in exemplary embodiments to weigh cost components in a final glucose cost function value.

### DETAILED DESCRIPTION

One problem suffered by many conventional insulin delivery devices is that low level glucose excursions above a target glucose level (“positive low level glucose excursions”) are persistent. The control algorithm used by the control system does not readily remove such positive low level glucose excursions. The positive low level glucose excursions may persist for long periods. Such persistent positive low level glucose excursions are not desirable and may have a negative effect on a user's health. The persistent positive low level glucose excursions are a product of how a cost function for a control loop of the insulin delivery device is formulated. The control loop seeks to deliver insulin dosages that minimize cost. The cost function conventionally is configured to be conservative and is not aggressive as to such persistent positive low level glucose excursions.

The exemplary embodiments may modify a glucose cost component of the cost function of the control loop of an insulin delivery device to compensate for persistent positive low level glucose excursions relative to a target glucose level. The exemplary embodiments may apply different functions for the glucose cost component depending on the current reading of glucose level of the user. For example, a linear glucose cost component function rather than a quadratic glucose cost component may be employed closer to the glucose level target for the user. The linear glucose cost component function more aggressively punishes positive low level glucose excursions than a quadratic glucose cost component function. The quadratic glucose cost component function is better suited for punishing more significant positive glucose excursions relative to the glucose level target for the user.

The exemplary embodiments may enable use of different glucose cost component functions for different glucose levels of the user. These glucose cost component functions may be employed in piecewise fashion with a different piece being applied for each respective range of glucose level values for the user. Thus, an aggerate glucose cost component for a user may include separate glucose cost component functions that are each applied only if the glucose level of the user is in the range associated with the respective glucose cost component function. The glucose cost component functions may be, for example, linear functions, quadratic functions, exponential functions, logarithmic functions, etc.

The final glucose cost function for calculating the glucose cost component may be a weighted combination of a piecewise glucose cost function and a weighted standard cost function (such as a quadratic function). The weights may reflect the magnitude and/or persistence of glucose excursions relative to a target glucose level. The persistence and/or magnitude of the positive excursions are captured in an offset value. The offset value then may be used to calculate the weights of the cost functions. The piecewise function helps to more aggressively reduce positive low-level glucose excursions than how aggressively a standard single type of cost function reduces the low-level glucose excursions. The piecewise cost function is more heavily weighted when the glucose excursions are more persistent and of greater magnitude. Thus, the weights dynamically adjust the balance between the piecewise cost function and the standard cost function based on the glucose excursion history.

The exemplary embodiments are not limited to insulin delivery devices but more broadly encompass medicament delivery devices that seek to keep an analyte level of a user at a target level. As will be elaborated upon below, the medicament is not limited to insulin but rather may be any of a wide variety of medicaments. Further, the analyte level need not be a glucose level of a user. Other analyte levels such as heart rate, body temperature, blood pressure, hormonal levels, respiration rate, etc. may be measured and used by the control loop. Examples of insulin delivery devices will be detailed below but are intended to be illustrative and not limiting.

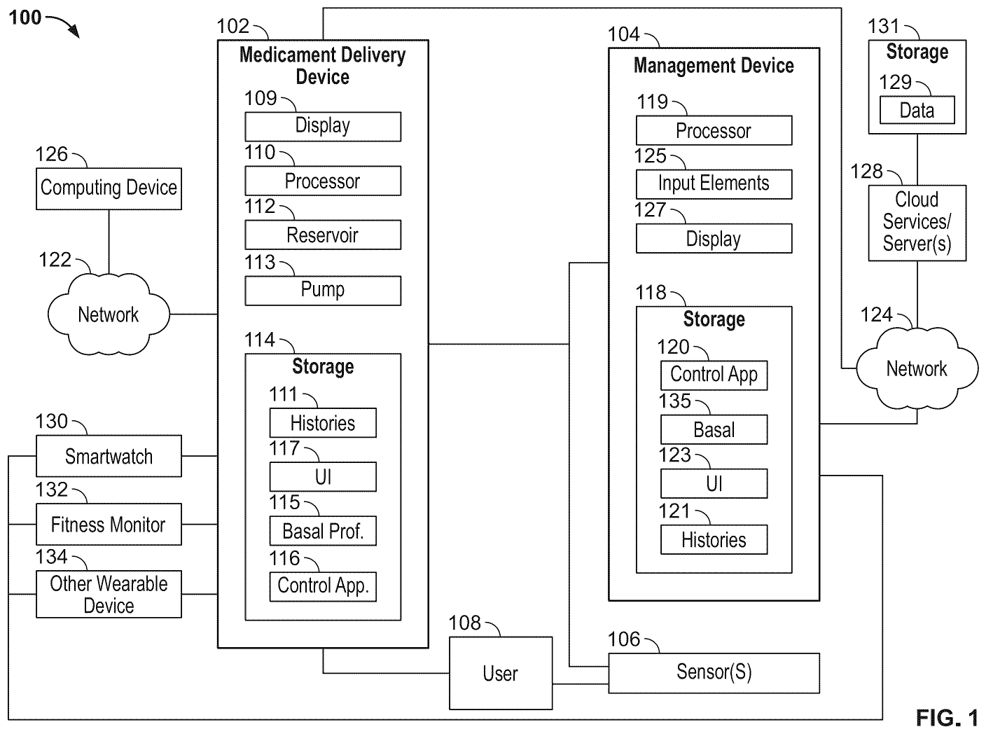

FIG. 1 depicts an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) or carried by the user (e.g., on a belt or in a pocket) with the medicament delivery device 102 being connected to an infusion site where the medicament is injected using a needle and/or cannula. In a preferred embodiment, a surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries, or the like. The control application may be responsible for implementing the control loop that provides feedback and adjustments to medicament dosages that are delivered to a user. The processor 110 also may execute computer programming instructions stored in the storage 114 for a user interface 117 that may include one or more display screens shown on display 109. The display 109 may display information to the user 108 and, in some instances, may receive input from the user 108, such as when the display 109 is a touchscreen.

The control application 116 may control delivery of a medicament to the user 108 per a control approach like that described herein. The storage 114 may hold histories 111 for a user, such as a history of basal deliveries, a history of bolus deliveries, and/or other histories, such as a meal event history, exercise event history, glucose level history and/or the like. These histories may be processed as will be described below to adjust basal medicament dosages to help reduce or eliminate persistent positive low level medicament excursions. The storage 114 also may include one or more basal profiles 115 that are used when the medicament delivery device is operating in open loop mode. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include one or more housings for housing its various components including a pump 113, a power source (not shown), and a reservoir 112 for storing a medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112) and may include a conduit to a separate infusion site.

There may be one or more communications links with one or more devices physically separated from the medicament delivery device 102 including, for example, a management device 104 of the user and/or a caregiver of the user, a sensor 106, a smartwatch 130, a fitness monitor 132 and/or another variety of wearable device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof.

The medicament delivery system 100 may or may not also include a management device 104. In some embodiments, no management device is not needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensors 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The one or more control applications 120 may be responsible for controlling the medicament delivery device 102, such as by controlling the AID delivery of insulin to the user 108. The storage 118 may store the one or more control applications 120, histories 121 like those described above for the medicament delivery device 102, one or more basal profiles 135 and other data and/or programs.

A display 127, such as a touchscreen, may be provided for displaying information. The display 127 may display user interface (UI) 123. The display 127 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128. The cloud services/server(s) 128 may provide output from the model 115 as needed to the management device 104 and/or medicament delivery device 102 during operation.

Other devices, like smartwatch 130, fitness monitor 132 and wearable device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

A wide variety of medicaments may be delivered by the medicament delivery device 102. The medicament may be insulin for treating diabetes. The medicament may be glucagon for raising a user's glucose level. The medicament may also be a glucagon-like peptide (GLP)-1 receptor agonists for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agents, a nutritional supplement, a vitamin, or co-formulations of two or more of the above.

The functionality described below for the exemplary embodiments may be under the control of or performed by the control application 116 of the medicament delivery device 102 or the control application 120 of the management device 104. In some embodiments, the functionality may be under the control of or performed by the cloud services or servers 128, the computing device 126 or by the other enumerated devices, including smartwatch 130, fitness monitor 132 or another wearable device 134.

The medicament delivery device 102 may operate in an open loop mode and in a closed loop mode. In the open loop mode, the user 108 manually inputs the amount of medicament to be delivered (such as per hour) for segments of the day. The inputs may be stored in a basal profile 115, 135 for the user 108. In other embodiments, a basal profile may not be used. The control application 116, 120 uses the input information from the basal profile 115, 135 to control basal medicament deliveries in open loop mode. In contrast, in the closed loop mode, the control application 116, 120 determines the medicant delivery amount for the user 108 on an ongoing basis based on a feedback loop. For an insulin delivery device, the aim of the closed loop mode is to have the user's glucose level at a target glucose level. The basal dosages may be delivered at fixed regular intervals, designated as cycles, such as every five minutes.

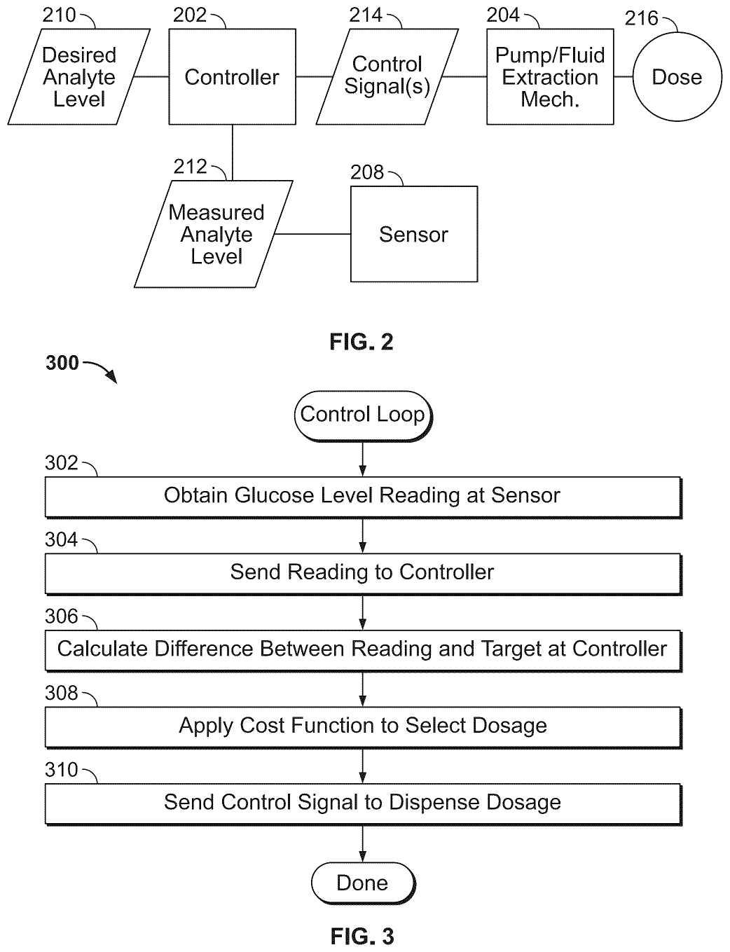

As was mentioned above, a control loop may be provided to adjust the basal delivery dosage based on current analyte level readings, such as glucose level readings, for example. FIG. 2 illustrates a simplified block diagram of an example of such a control loop 200 suitable for practicing an exemplary embodiment. The example control loop 200 may include a controller 202, a pump mechanism or other fluid extraction mechanism 204 (hereinafter “pump 204”), and a sensor 208. The controller 202, pump 204, and sensor 208 may be communicatively coupled to one another via a wired or wireless communication paths. The sensor 208 may be a glucose monitor in some exemplary embodiments, such as, for example, a continuous glucose monitor (CGM). The sensor 208 may, for example, be operable to measure glucose level values of a user to generate the measured analyte level 212.

As shown in the example, the controller 202 may receive a desired analyte level 210, indicating a desired analyte level or range for a user. The desired analyte level 210 may be received from a user interface to the controller 202 or other device or by an algorithm that automatically determines a desired analyte level 210 for a user. The sensor 208 may be coupled to the user and be operable to measure an approximate value of an actual analyte level of the user. For cases where the analyte level is a glucose level, it is worth noting that the measured glucose level is only an approximate value of a user's glucose level. There may be errors in the measured glucose levels. The errors may, for example, be attributable to factors, such as age of the sensor 208, location of the sensor 208 on a body of a user, environmental factors (e.g., altitude, humidity, barometric pressure), or the like. In response to the measured analyte level or value, the sensor 208 may generate a signal indicating the measured analyte level 212. The controller 202 may receive from the sensor 208 via a communication path the measured analyte level signal 212.

Based on the desired analyte level signal 210 and the measured analyte level signal 212, the controller 202 may generate one or more control signals 214 for directing operation of the pump 204. For example, one of the control signals 214 may cause the pump 204 to deliver a dose of medicament 216 to a user via output 206. The dose of medicament 216 may, for example, be determined based on a difference between the desired analyte level 210 and the actual analyte level 212. The cost function referenced above plays a role in determining the dosage as part of the closed loop control system as will be described below. The dose of medicament 216 may be determined as an appropriate amount of medicament to drive the actual analyte level of the user toward the desired glucose level. Based on operation of the pump 204 as determined by the control signals 214, the user may receive the dose of medicament 216 from the pump 204.

FIG. 3 depicts a flowchart 300 of steps that may be performed by exemplary embodiments in determining what dose of medicament to deliver the user as part of the closed loop control system. These steps may be performed by controller 110, processor 119 or other components (at least in part), like smartwatch 130, fitness monitor or wearable device 134. That said, for purposes of simplicity below, the discussion just refers to controller 110. Initially, as was described above relative to FIG. 2, a glucose level reading is obtained by the sensor 208 at 302. The analyte level reading is sent via a signal 212 to the controller 202 at 304. The controller 202 calculates an error value as the difference between the measured analyte level 212 and the desired analyte level 210 at 306. The closed loop control system attempts to minimize the aggregate penalty of the cost function over a wide range of possible dosages. The cost function is applied to the possible dosages, and the dosage with the best cost function value is selected at 308. Depending on how the cost function is configured, the best value may be the lowest value or the highest value. The cost function used in exemplary embodiments will be described in more below. A control signal 214 may be generated by the controller 202 and sent to the pump 204 to cause the pump to deliver the desired medicament dose 216 to the user at 310.

As mentioned above, the medicament may be insulin and the analyte level sensed by the sensor(s) 106 may be glucose level. The cost function may be adjusted to address persistent low-level glucose level excursions for users. As a starting point for this discussion, it is helpful to review an exemplary cost function. An exemplary formulation for cost J is:

$$J = Q \cdot {\sum }_{i = 1}^{M} {{G}_{p} ( i )}^{2} + R \cdot {\sum }_{i = 1}^{n} {{I}_{p} ( i )}^{2}$$

where Q and R are weight coefficients as mentioned above, Gₚ(i)² is the square of the deviation between the projected glucose level for an insulin dosage at cycle i and the projected glucose level for the basal insulin dosage, M is the number of cycles in the prediction horizon, Iₚ(i)² is the square of the deviation between the projected insulin delivered at cycle i and the insulin for basal insulin delivery, and n is the control horizon in cycles. Thus,

$$Q \cdot {\sum }_{i = 1}^{M} {{G}_{p} ( i )}^{2}$$

is the weighted glucose cost, and

$$R \cdot {\sum }_{i = 1}^{n} {{I}_{p} ( i )}^{2}$$

is the weighted insulin cost. The total cost J is the sum of the weighted glucose cost and the weighted insulin cost. A cycle has a fixed interval, such 5 minutes.

The exemplary embodiments may modify the glucose cost component of the cost function to compensate for low level glucose excursions relative to a target glucose level. More generally, the exemplary embodiments may apply different functions for the glucose cost component depending on the current reading of glucose level of the user. For example, a linear glucose cost component function rather than a quadratic glucose cost component may be employed closer to the glucose level target for the user. The linear glucose cost component function more aggressively punishes low level glucose excursions than a quadratic glucose cost component function does. The quadratic glucose cost component function is better suited for punishing more significant glucose excursions relative to the glucose level target for the user.

The exemplary embodiments may enable use of different glucose cost component functions for different glucose levels of the user. These glucose cost component functions may be employed in piecewise fashion with a different piece being applied for each respective range of glucose level values for the user. Thus, an aggerate glucose cost component for a user may be includes separate glucose cost component functions that are each applied only if the glucose level of the user is in the range associated with the respective glucose cost component functions. The functions may be, for example, linear functions, quadratic functions, exponential functions, logarithmic functions, etc.

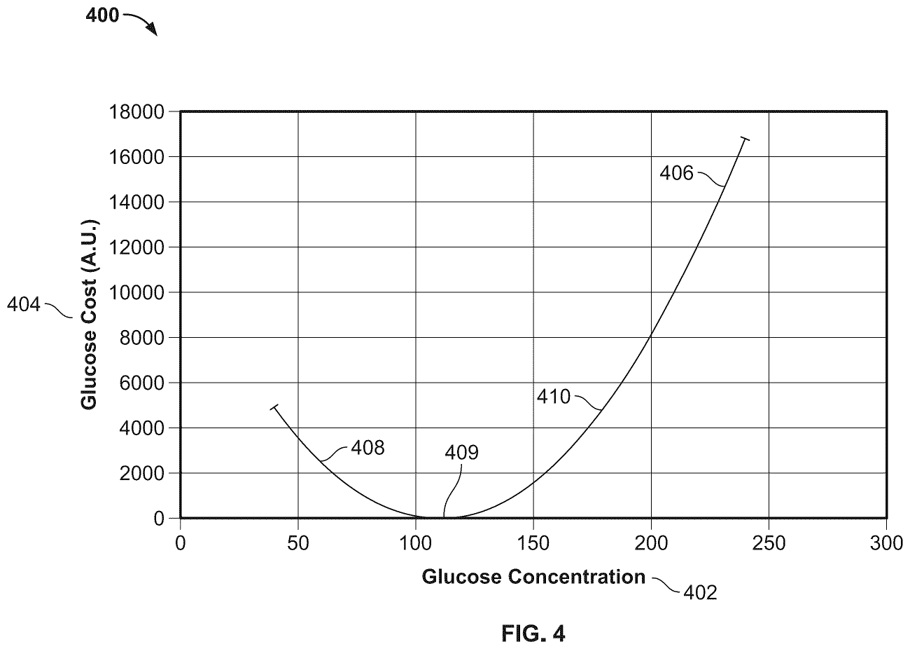

FIG. 4 depicts a graph 400 of an illustrative quadratic glucose cost component that may be used in a control loop of an insulin delivery device. The graph plots a curve 406 of glucose concentration 402 of a user relative to glucose cost 404. The glucose concentration 402 is expressed as mg/dL and the glucose cost is expressed in arbitrary units (A.U.). The curve extends from a glucose concentration of approximately 40 mg/dL to approximately 240 mg/dL. It is presumed that the target glucose concentration 409 for the user is 110 mg/dL. Other target concentrations may be used. The desired range of glucose values is between the hypoglycemic threshold of 70 mg/dL and the hyperglycemic threshold of 180 mg/dL. The slope of curve 406 is negative when the user's glucose concentration is below the target glucose concentration 409 of 110 mg/dL. Thus, the glucose cost 404 drops until the target glucose concentration 409 is reached. In section 408 of the curve 406, the slope starts off greater and lessens as the target glucose concentration is reached. The result is that section 408 flattens out near the target glucose concentration 409. Hence, the quadratic function decreases insulin delivery more aggressively when glucose concentration is below the hypoglycemic threshold of 70 mg/dL, but is less aggressive in its reduction of insulin delivery as the target glucose concentration 409 is reached (going from left to right).

With the quadratic function curve 406 of FIG. 4, the slope of the curve reaches 0 at the target glucose concentration 409 and become positive thereafter. Thus, the slope of section 410 of the quadratic function curve 406 is positive. The positive curve indicates that the glucose cost increases as the glucose concentration increases. The slope of section 410 starts off modestly near the target glucose concentration 409 and increases. After 180 mg/dL, the slope becomes quite great to encourage more insulin delivery as the glucose cost is high. The helps to discourage the user from becoming hyperglycemic.

One drawback of using a quadratic glucose cost function like that shown in FIG. 4 is that it is difficult to get rid of positive low level glucose excursions. The glucose cost for such excursions is negligible as indicated by the flatness of the curve 406 immediately to the right and left of the target glucose concentration. Hence, the control loop using such a quadratic glucose cost function is not punished much for such positive low level glucose excursion and as a result, is not especially effective in eliminating them.

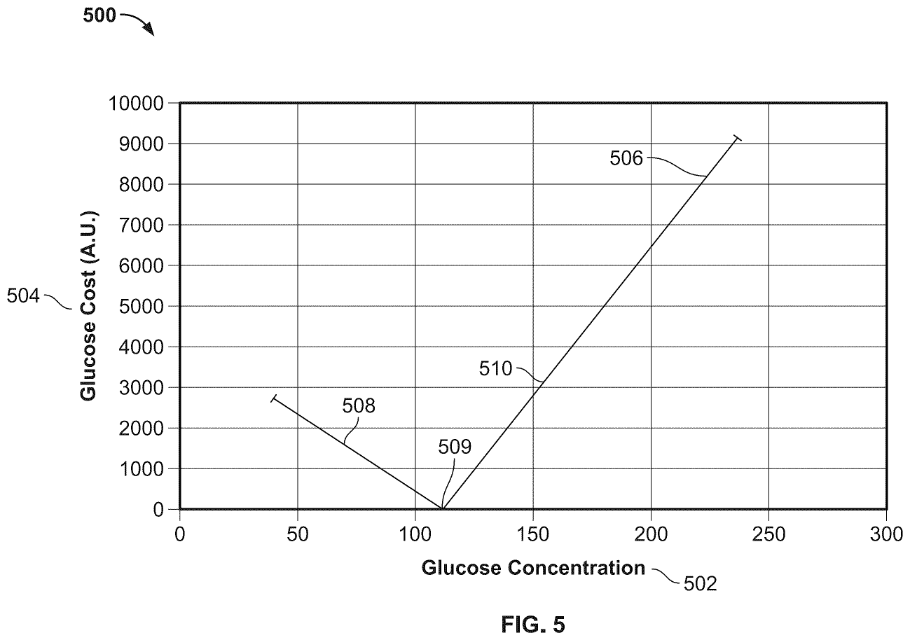

FIG. 5 depicts a plot 500 of a curve 506 of linear glucose cost function showing glucose concentration 502 versus glucose cost. The curve 506 has a portion 508 with a negative slope extending between 40 mg/dL and the target glucose concentration 509 of 110 mg/dL. Thus, when the glucose concentration 502 is below the target glucose concentration 509, the glucose cost decreases until the target glucose concentration 509 is reached. As the glucose concentration 502 increases above the target glucose concentration 509 in portion 510 of the curve 506, the slope of the curve is positive and constant. This indicates that the glucose cost 504 increases linearly as the glucose concentration 502 increases above the target glucose concentration.

The exemplary embodiments may combine analyte level cost functions. For instance, a first analyte level cost function may be used for a first range of analyte levels and a second analyte cost function may be used for a second range of analyte levels. In other instances, more than two analyte level cost functions may be used. Such analyte level cost functions that combine multiple analyte level cost functions are referred to herein as “piecewise functions.”

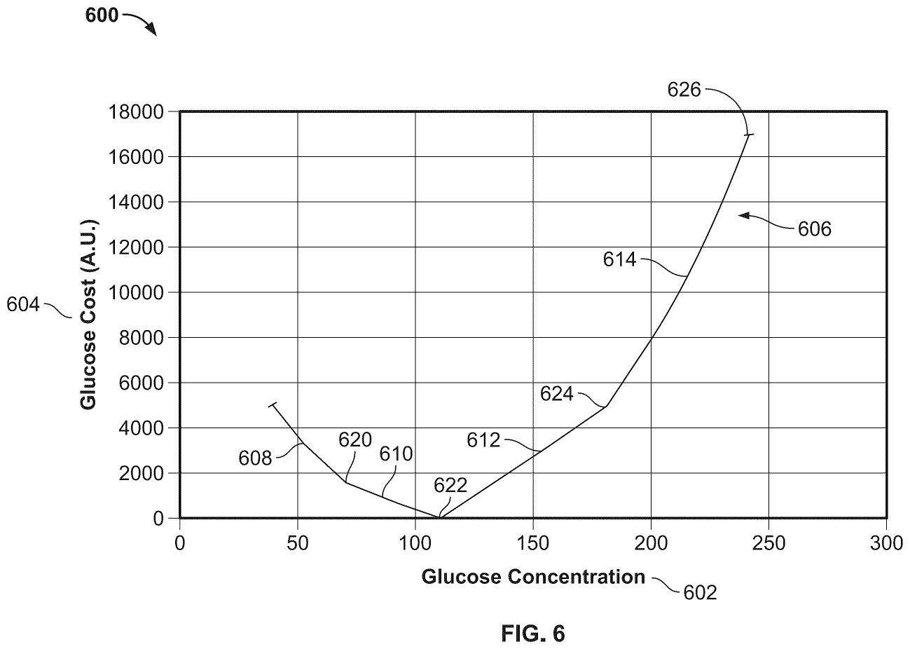

For an insulin delivery device, the exemplary embodiments may combine multiple glucose cost functions into a piecewise glucose cost function. FIG. 6 depicts an example of a piecewise glucose cost function wherein a linear cost function is combined with a quadratic cost function. FIG. 6 shows a graph 600 of the curve 606 of glucose concentration 602 relative to glucose cost 604 for the piecewise glucose cost function. In this piecewise glucose cost function, the quadratic function is used for values below the hypoglycemic threshold 620 of 70 mg/dL as indicated by portion 608 that extends from 40 mg/dL to 70 mg/dL. The quadratic cost function is also used for glucose concentration 602 values above the hyperglycemic threshold 624 of 180 mg/dL in portion 614 of the curve 606. The quadratic cost function is suitable for when the glucose concentration 602 of the user is below the hypoglycemic threshold 620 to increase the glucose cost 604 rapidly. The quadratic function also is suitable when the glucose concentration 602 of the user is above the hyperglycemic threshold 624 because it is desirable to increase the glucose cost 604 rapidly to discourage additional increases in glucose concentration 602.

A linear cost function is used for portion 610 of the curve 606 between the hypoglycemic threshold 620 and the target glucose concentration 622. The linear cost function increases the glucose cost more rapidly than the quadratic cost function in this portion 610. A linear cost function is used for the portion 612 of the curve 606 between the target glucose concentration 622 and the hyperglycemic threshold 624. The linear function increases the glucose cost 604 more rapidly than the quadratic glucose cost function for this range of glucose concentration values and thus more aggressively reduces low level positive glucose excursions.

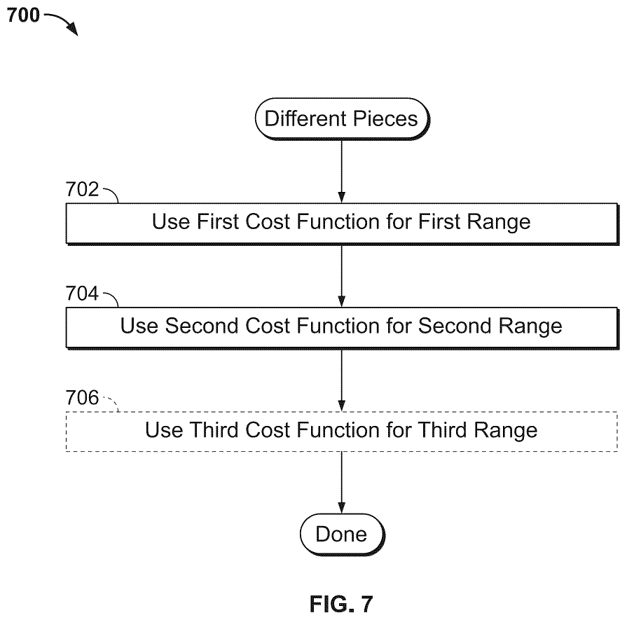

FIG. 7 depicts a flowchart 700 of illustrative steps that may be performed in exemplary embodiments to use a piecewise analyte level cost function. At 702, the piecewise cost function uses a first analyte level cost function (e.g., a linear glucose cost function) for a first range of analyte levels for a user. At 704, the piecewise cost function uses a second analyte level cost function (e.g., a quadratic glucose cost function) for a second range of analyte levels for the user. The piecewise analyte level cost function may use more than two cost functions to determine the costs of additional ranges of analyte levels. Any number of analyte level cost functions may be included in the piecewise analyte level cost function. At 706, an optional third analyte level cost function may be used for a third range of analyte levels. This step 706 is shown to illustrate that more than two analyte cost functions may be used in the piecewise analyte level cost function.

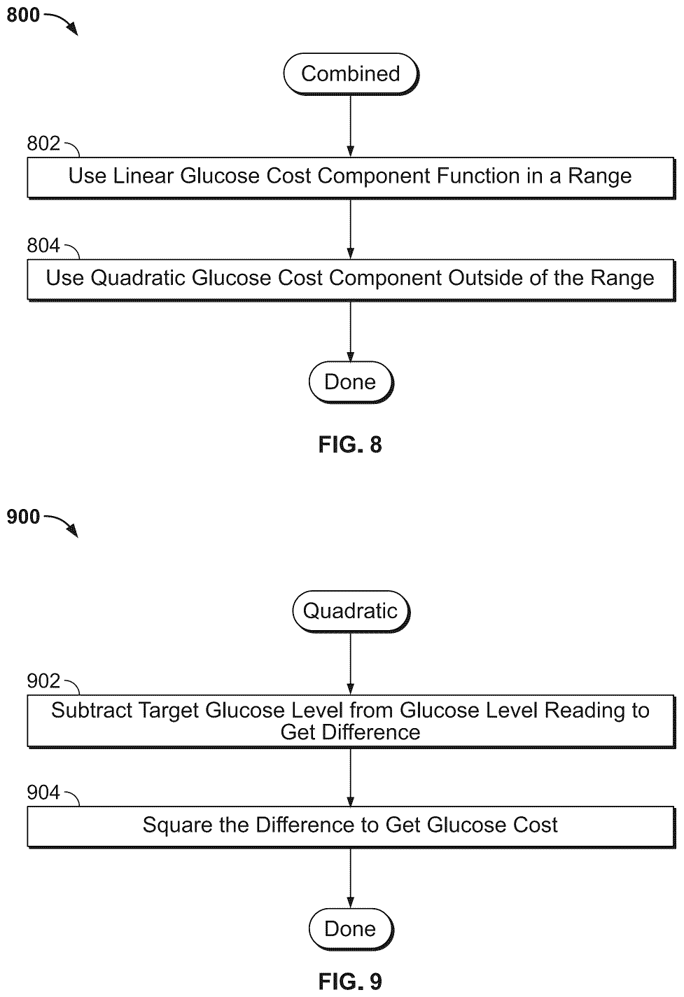

The use of such a piecewise analyte level function may be used by a medicament delivery device that delivers insulin to a user. In such a case, the glucose level of the user is the analyte level that is sensed and is used in the control loop. In the exemplary embodiments, a linear glucose cost function and a quadratic glucose cost component may be used as part of a piecewise glucose cost component. As shown in the flowchart 800 of FIG. 8, at 802, a linear cost component function may be used for a range of glucose values for the user. As shown in the example of FIG. 6, a linear cost function is used for glucose concentration 602 values between the hypoglycemic threshold 620 of 70 mg/dL and the hyperglycemic threshold 624 of 180 mg/dL. This range includes portions 610 and 612 of the piecewise cost function curve 606. At 804, a quadratic glucose cost function is used outside of the range where the linear glucose cost function is used. In the example of FIG. 6, the quadratic glucose cost function is used for glucose concentration 602 values below the hypoglycemic threshold 620 of 70 mg/dL and above the hyperglycemic threshold 624 of 180 mg/dL. Other ranges may be used with the respective cost function. As was discussed above relative to FIG. 6, this approach increases aggressiveness where needed and reduces aggressiveness where needed. This piecewise approach helps to reduce the persistent positive low level glucose excursions.

A suitable quadratic glucose cost function is:

$${J}_{quad} ( i ) = {( G ( i ) - SP ( i ) )}^{2} \qquad ( Equation 1 )$$

where G(i) is the glucose level of the user at cycle i, and SP(i) is the target glucose level (e.g., concentration) for the user. FIG. 9 depicts a flowchart 900 of illustrative steps that may be performed in exemplary embodiments to determine a glucose cost using Equation 1 for a cycle i. At 902, the target glucose level (i.e., SP(i)) is subtracted from the glucose level at cycle i (i.e., G(i)). At 904, the resulting difference is squared. Thus, the glucose cost is the square of the difference between the user's current glucose value and the target glucose value.

A suitable linear glucose cost function to be used in the exemplary embodiments in a piecewise glucose cost function is:

$$( Equation 2 )$$

$${J}_{linear} ( i ) = ( SP ( i ) - 70 ) \cdot \vert {G}_{< SP ( i )} ( i ) - SP ( i ) \vert + ( 1 \; 80 - SP ( i ) ) \vert {G}_{\geq SP ( i )} ( i ) - SP ( i ) \vert .$$

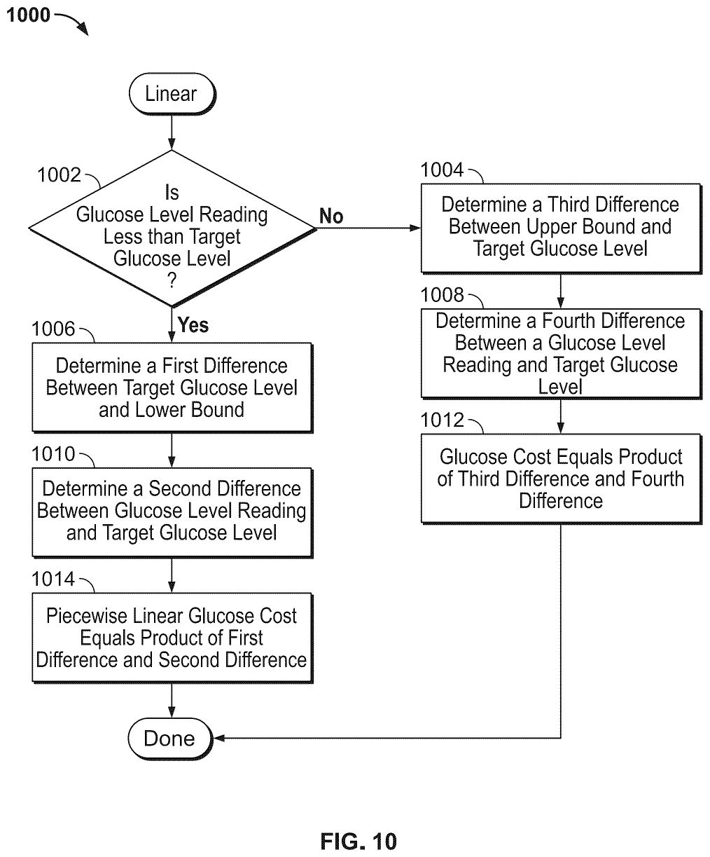

FIG. 10 depicts a flowchart 1000 of illustrative steps that may be performed to determine the linear glucose cost at cycle i using Equation 2. At 1002, a check is made whether the glucose level of the user G(i) is less than the target glucose level SP(i). If it is, at 1006, a first difference between the target glucose level and the lower bound (i.e., the hypoglycemic threshold of 70 mg/dL) is determined (i.e., (SP(i)−70)). At 1010, a second difference between the glucose level reading and the target glucose level is determined (i.e., G_{<SP(i)}(i)−SP(i)). At 1014, the piecewise linear glucose cost (i.e., Jₗᵢₙₑₐᵣ(i)) is calculated as the product of the first difference and the second difference. The other portion of Equation 2 (i.e., (180−SP(i))|G_{≥SP(i)}(i)−SP(i)|) is 0 as the glucose level of the user is not greater than or equal to the target glucose level.

If at 1002, the glucose level reading is not less than the target glucose level, a third difference between an upper bound (i.e., the hyperglycemic threshold) and the target glucose level is determined (i.e., 180−SP(i)). A fourth difference between the current glucose level reading of the user and the target glucose level is determined (i.e., G_{≥SP(i)}(i)−SP(i)). At 1012, the piecewise linear glucose cost is determined to be the produce of the third difference and the fourth difference (i.e., (180−SP(i))|G_{≥SP(i)}(i)−SP(i)|).

The combined piecewise glucose cost function, which combines the linear glucose cost function and the quadratic glucose cost function, may be expressed as:

$$( Equation 3 )$$

$${J}_{piecewise} ( i ) = ( SP ( i ) - 70 ) \cdot \vert {G}_{70 \leq G < SP ( i )} ( i ) - SP ( i ) \vert + ( 1 \; 80 - SP ( i ) ) \vert {G}_{SP ( i ) \leq G \leq 180} ( i ) - SP ( i ) \vert + {( {G}_{< 70} ( i ) - SP ( i ) )}^{2} + {( {G}_{> 180} ( i ) - SP ( i ) )}^{2} .$$

It should be appreciated that other formulations of the cost functions may be used. For example different weights may be used and the ranges where the cost functions are applied may be different than the above examples.

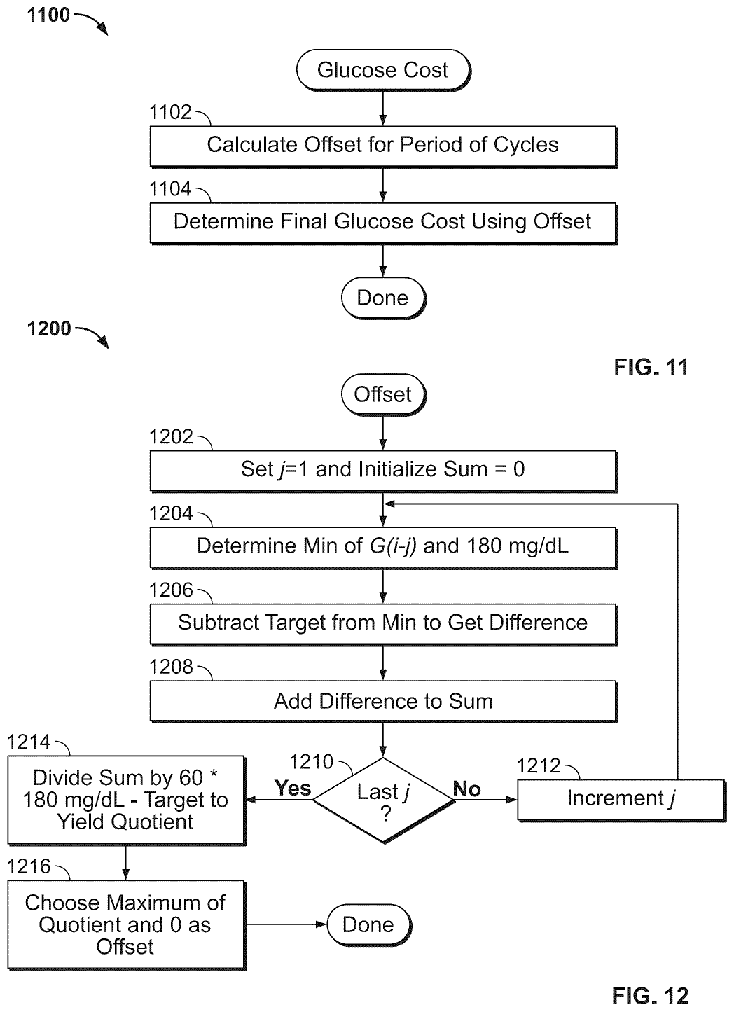

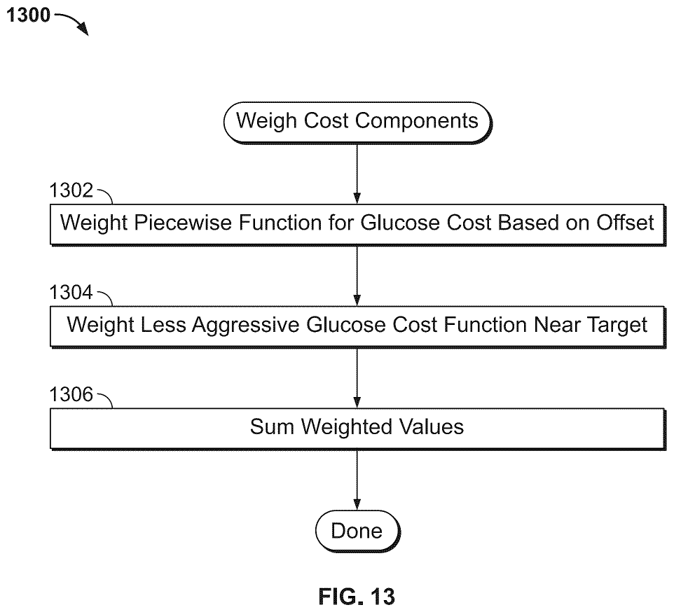

As mentioned above, the glucose cost function may be further adjusted to attempt to better eliminate persistent positive low level glucose excursions. The use of the piecewise function J_{piecewise}(i) may be weighted based on how persistent the glucose excursions historically are and the magnitude of such persistent positive low level glucose excursions. In this regard, the exemplary embodiments may perform the illustrative steps depicted in the flowchart 1100 of FIG. 11. First, at 1102, an offset is calculated. The offset is a value between 0 and 1 that represents how often and how much the glucose level of the user exceeded the threshold over a recent period (that data of which may be found in the histories 111).

One suitable formula for calculating the offset in exemplary embodiments is:

$${Q}_{offset} ( i ) = max ( \frac{{\sum }_{j = 1}^{60} min ( G ( i - j ) , 180 ) - SP ( i - j )}{60 \cdot ( 180 - SP ( i - j ) )} , 0 ) \qquad ( Equation 4 )$$

where max( ) is a function that returns a maximum of values and min( ) is a function that returns a minimum of values. FIG. 12 depicts a flowchart 1200 of illustrative steps that may be performed in exemplary embodiments to calculate the offset per Equation 4. At 1202, the variable j is initialized to have a value of 1 and

$${\sum }_{j = 1}^{60} min ( G ( i - j ) , 180 ) - SP ( i - j )$$

is initialized as 0. The index j is incremented from 1 to 60 in the summation as the period for which the values are aggregated is the 60 cycles prior to the current cycle i. Where each cycle is 5 minutes, the 60 cycles constitute a 5-hour period. Other lengths for the period may be used as well. At 1204, the minimum of G(i−j) and 180 is determined. This ensures that values greater than 180 are not selected. The value 180 constitutes the hyperglycemic threshold of 180 mg/dL. The units have not been explicitly recited in the equation. At 1206, a difference is calculated by subtracting the target glucose value from the minimum. At 1208, the difference is added to the aggregated sum. A check is made if j is the last j (e.g., 60). If not, at 1212, j is incremented and the process repeats beginning at 1204. The process repeats until all 60 cycles are processed. The resulting summation represents the aggregate sum of the differences with the maximum glucose value being 180.

When checked at 1210, if j=60, at 1214, the aggregate sum is divided by 60 times (180 mg/dL−the target glucose level). This produces a value indicative of the average amount that the glucose level of the user exceeds the target glucose level relative to the difference being (180 mg/dL−the target glucose level) each cycle of the period. At 1216, the maximum of the quotient calculated in 1214 and 0 is chosen as the quotient. Choosing the maximum eliminates negative values.

With reference to FIG. 11 again, once the offset has been calculated at 1102, the offset may be used to determine the final glucose cost. In one exemplary case, the final glucose cost may be calculated as:

$${J}_{final} ( i ) = ( 1 - {Q}_{offset} ( i ) ) \cdot {J}_{quad} ( i ) + {Q}_{offset} ( i ) \cdot {J}_{piecewise} ( i ) . \qquad ( Equation 5 )$$

With this equation, the proportion of the final glucose cost function that utilizes the linear cost between 70-180 mg/dL versus the quadratic cost between 70-180 mg/dL can be determined based on the Q_{offset} to allow the cost function to scale more rapidly with lower glucose excursions above the target glucose level. Specifically, the cost due to the linear cost function is more heavily weighted the higher the Q_{offset}, representing more persistent hyperglycemia.

FIG. 130 depicts a flowchart 1300 of illustrative steps that may be performed in exemplary embodiments to calculate the final glucose cost using the offset. At 1302, the weight applied to the piecewise function for glucose cost is determined. In Equation 5, the weight is the offset Q_{offset}(i). At 1304, a weight is applied to the less aggressive glucose cost function near the target glucose value. In Equation 5, the quadratic glucose cost function is the less aggressive glucose cost function near the target glucose value. The weight in Equation 5 that is applied is (1−Q_{offset}(i)). At 1306, the weighted values are summed as in Equation 5.

While the application has described with reference to exemplary embodiments herein, it should be appreciated that various changes in form and detail relative to the exemplary embodiments may be made without departing from the intended scope as defined by the appended claims.

1. A medicament delivery system, comprising:
a non-transitory storage medium for storing computer programming instructions for controlling operation of the medicament delivery system;
a processor for executing the computer programming instructions to cause the processor to:
determine a lowest cost dose of medicament in a set of candidate doses of medicament for a user by calculating a cost for each candidate dose in the set of candidate doses of the medicament;
wherein each calculated cost includes a medicament cost component and an analyte cost component for the candidate dose;
wherein the analyte cost component is determined by selecting among analyte cost functions based on a most recent glucose level of the user and using the selected analyte cost function to determine the analyte cost component; and
determine that a next medicament dose for delivery from the medicament delivery system is the lowest cost dose.

2. The medicament delivery system of claim 1, wherein a first analyte cost function is used in determining the analyte cost component where the most recent glucose level of the user is below a threshold and a second analyte cost function is used in determining the analyte cost component where the most recent glucose level of the user is above the threshold.

3. The medicament delivery system of claim 2, wherein the first analyte cost function is a quadratic function.

4. The medicament delivery system of claim 3, wherein the second analyte cost function is a linear function.

5. The medicament delivery system of claim 3, wherein the second analyte cost function is an exponential function.

6. The medicament delivery system of claim 2, wherein the first analyte cost function is an exponential function or a logarithmic function.

7. The medicament delivery system of claim 2, wherein the threshold is a target glucose level of the user.

8. The medicament delivery system of claim 1, wherein the medicament is insulin and wherein the analyte cost is a glucose cost.

9. The medicament delivery system of claim 1, wherein the selected analyte cost function is a piecewise function that combines multiple analyte cost functions.

10. A medicament delivery system, comprising:
a non-transitory storage medium for storing computer programming instructions for controlling operation of the medicament delivery system;
a processor for executing the computer programming instructions to cause the processor to:
determine a lowest cost dose of medicament in a set of candidate doses of medicament for a user by calculating a cost for each candidate dose in the set of candidate doses of the medicament;
wherein each calculated cost includes a medicament cost component and an analyte cost component for the candidate dose; and
wherein the analyte cost component is determined by selecting among analyte cost functions based on a most recent glucose level of the user and using the selected analyte cost function to determine the analyte cost component such that a first analyte cost function is used when the most recent glucose level of the user is in a first range, a second analyte cost function is used when the most recent glucose level of the user is in a second range, and a third analyte cost function is used when the most recent glucose level of the user is in a third range; and
determine that a next medicament dose for delivery from the medicament delivery system is the lowest cost dose.

11. The medicament delivery system of claim 10, wherein the selected analyte cost function is a combination of a piecewise cost function and an additional cost function.

12. The medicament delivery system of claim 11, wherein weights assigned to the piecewise cost function and the additional cost function in the analyte cost function depend on persistence and magnitude of analyte excursions experienced by the user.

13. The medicament delivery system of claim 10, wherein the first analyte cost function, the second analyte cost function, and the third analyte coast function are each one of a quadratic function, an exponential function, a linear function, or a logarithmic function.

14. The medicament delivery system of claim 10, wherein the medicament comprises insulin.

15. A method performed by a processor of a medicament delivery system for controlling basal medicament deliveries by the medicament delivery device, comprising:
with the processor, determining a medicament cost for a candidate basal medicament dosage for delivery to a user by the medicament delivery device;
with the processor, determining an analyte cost for the candidate basal medicament dosage, the analyte cost comprising:
where an analyte level of the user in a prediction horizon if the candidate basal medicament dosage is delivered to the user is within a specified range of a target analyte level of the user, a value produced by a first type of function of a deviation between the analyte level of the user in the prediction horizon and the target analyte level of the user, wherein the first type of function is one of, a linear function type, a quadratic function type, a logarithmic function type, or an exponential function type;
where a analyte level of the user in the prediction horizon if the candidate basal medicament dosage is delivered to the user is outside a specified range of a target analyte level of the user, a value produced by a second type of function of a deviation between the analyte level in the prediction horizon and the target analyte level of the user, wherein the second type of function differs from the first type of function and the second type of function is one of, a linear function type, a quadratic function type, a logarithmic function type, or an exponential function type;
with the processor, determining a cost for the candidate basal medicament dosage using the medicament cost and the analyte cost; and
with the processor, based on the cost, deciding to deliver the candidate basal medicament dosage.

16. The method of claim 15, wherein the medicament is insulin.

17. The method of claim 15, further comprising weighting the analyte cost in the determining of the cost for the candidate basal medicament dosage.

18. The method of claim 17, further comprising weighting the medicament cost in the determining of the cost for the candidate basal medicament dosage.

19. The method of claim 17, wherein the cost for the candidate basal medicament dosage is the sum of the weighted medicament cost and the weighted analyte cost.

20. The method of claim 15, wherein the medicament delivery system includes a medicament delivery device and a management device for managing the medicament delivery device.
